(19) United States
(12) Patent Application Publication
(10) Pub. No.: US 2011/0286935 A1
(43) Pub. Date: Nov. 24, 2011

(54) Multi-Functional Nano-Device

(75) Inventors: Patrick McCarthy, Pittsburgh, PA (US); James Spanswick, Pittsburgh, PA (US)

(73) Assignee: ATRP Solutions, Inc., Pittsburgh, PA (US)

(21) Appl. No.: 13/109,256

(22) Filed: May 17, 2011

Related U.S. Application Data
(60) Provisional application No. 61/395,908, filed on May 19, 2010.

(51) Int. Cl. A61K 49/18; A61K 39/395; A61P 35/00; A61K 9/00; A61K 49/00; B82Y 15/00; B82Y 5/00

(52) U.S. Cl. 424/9.34; 424/400; 424/178.1; 424/9.1; 424/9.6; 977/700; 977/906; 977/927

(57) ABSTRACT

A universal drug delivery platform for monoclonal antibody-based therapeutics is described. This universal platform resolves the problems of immunogenic response associated with the present monoclonal antibody based therapeutics by providing a multifunctional nano-device which comprises a well defined core/shell nano-structure that can function as a drug delivery platform linked to a monoclonal antibody through a single linking group.

### CROSS REFERENCE TO RELATED APPLICATION

This application claims the benefit of U.S. Provisional Application 61/395,908, filed May 19, 2010. The foregoing related application, in its entirety, is incorporated herein by reference.

### FIELD OF THE INVENTION

A universal drug delivery platform for monoclonal antibody based therapeutics is described. This universal platform resolves the problems of immunogenic response associated with the present monoclonal antibody based therapeutics by providing a multifunctional nano-device which comprises a well defined core/shell nano-structure that can function as a drug delivery platform linked to a monoclonal antibody through a single linking group.

### BACKGROUND OF THE INVENTION

Monoclonal antibodies, (MAb), and fragments thereof, are emerging as one of the dominant classes of protein based therapeutics. However several problems are emerging within the MAb therapeutics industry. Patients are experiencing side effects, resulting from immunogenic response to the treatment, and hospital technicians are having difficulties preparing and administering MAb based drugs.

A typical MAb treatment involves administering 50-100 mg of monoclonal antibody per treatment. (J. Pharm. Sci., 2005, 93, 1390-1402.) Since MAbs cannot be delivered orally, administration is typically achieved by injection. MAb therapeutics, are delivered to hospitals and stored as solids in single dose vials. A technician at the hospital dissolves the drug in a solvent prior to injection. (Adv. Drug Deliv. Rev., 2006, 58, 686-706.) For example, 50-100 mg of drug will be diluted with 1-2 mL of solvent to form a solution. This 50 mg/mL solution is viscous and has a short shelf life (Liu, J.; et al. J. Pharm. Sci. 2005, 94, 1928-1940.) The technician will then fill the syringe and treat the patient.

The human body has an immunogenic response when a foreign substance is detected. Recent studies have shown that the extent of the immunogenic response is correlated with the extent of MAb aggregation. (Rosenberg, A. S.; AAPS J 2006, 8, E501-E507.) MAb aggregation can occur during production of the drug, during drug solubilization, or during administration of the drug by driving a viscous solution through a narrow gauge syringe needle. There is a potential to reduce the extent of MAb aggregates delivered to the body by either reducing the required concentration of MAb within the formulation by or increasing the efficacy of the drug. Diluting the drug, however, results in a greater volume of liquid required for each treatment. This makes injection by a syringe cumbersome.

There are a variety of approaches the bio-pharmaceutical industry leaders are pursuing to alleviate this problem. One approach involves reduction in variability from solubilization and syringe needle type by providing hospitals with pre-filled syringes. This approach is facing its own set of problem due to short MAb shelf life.

Very few monomclonal antibodies are therapeutically useful since they only display modest cell killing ability. An approach to increase cytotoxicity is to reduce the amount of MAb needed for treatment by conjugating small molecule drugs directly to the MAb, thereby, increasing its efficacy and making it possible to inject smaller amounts of drug. As many as 10 small molecule drugs have been linked to a single antibody. However this does not alleviate problems with immunogenic response and the preferred drug loading is an average of four or less units per MAb are linked via lysine residues to the antibody. (Chari, R. V. J. Acc. Chem. Res. 2008, 41, 98-107; Hamann, P. R.; et. al., Bioconjugate Chem. 2005, 16, 354-360.) In addition linkage of more cytotoxic drugs is not possible because the drugs are usually hydrophobic and poorly soluble in predominately aqueous media and presence of the drugs may adversely alter the pharmakinetics of the antibody and diminish the binding affinity for the target cell.

An additional problem with the present MAb based treatments is that the scientists not only have to select the best antibody to carry the drug to its target but must chose a linker that keeps the drug tethered to the antibody in the blood but releases the drug once it reaches the tumor cells. This means that one of the major stumbling blocks of antibody conjugates is to design a linker that works successfully. Chari has noted that disulfide bond are effective linking groups but each drug molecule has to be modified and linkage to the monomclonal antibody is still a random linking event.

In the non-limiting exemplary multifunctional nano-device that functions as a drug delivery platform described herein this difficulty is circumvented by encapsulation of multiple drug molecules, preferentially ten or more drug molecules, in the core of the nano-device which is optionally attached to the monoclonal antibody by a more stable, but still biodegradable, linking group.

There are several research laboratories working on developing novel nano-scale imaging and drug delivery devices. Recent reviews by Ferrari (M. Ferrari, Nature Reviews Cancer, 2005, 5, 161.) and Polakis (Current Opinion in Pharmacology, 2005, 5(4), 382-387) indicate that significant progress in fundamental cancer biology in the previous 25 years has not translated into even distantly comparable advances in the clinic. The discrepancy could be attributed to inadequacies in the ability of physicians to administer therapeutic agents so that they can selectively reach the desired targets with marginal or no collateral damage. Ferrari also outlined the progress in generating multifunctional nanoparticles, or nanovectors, for drug delivery. A few of the more advanced devices made to date include devices made from shell crosslinked kneddles (SCKs), magnetic or imaging nanoparticles, and dendrimers, but as discussed below these advances still have inherent inadequacies.

SCKs: Work on SCKs has been carried out by Karen Wooley's laboratory at Washington University (Wooley, K. L., et. al., Macromolecules, 2004, 37(19), 7109-7115; and U.S. patent application Ser. No. 11/250,830 published as 20060159619) These SCK devices were fabricated by inducing block copolymers to self assemble into micelle like structures, then, these micellular units were crosslinked through the shell or the core resulting in formation of a stable nano-structure. Although these structures have been proven to have utility in a variety of application areas, this approach is not suitable for use as a targeted nano-device in this application for the following reasons. The ability to modify the size of a SCK's is limited, in that the self assembled micelles have limited variability in the size of the first formed micelle. In an SCK approach, the critical step is self assembly of the first formed micelle which limits the degree of control over the dimensions of the SCK and furthermore each crosslinked block copolymer within the SCK retains an available reaction site. An attempt to conjugate this rather large structure to a MAb would result in an SCK decorated with many MAb's. A device made in this way resembles a MAb aggregate and would most likely illicit an immunogenic response in the patient.

The device which we disclose herein is not easily accessible by an SCK approach.

The concept of the disclosed desired nano-device conjugate is that a single nano-device of predetermined dimensions and predetermined drug carrying capacity will be conjugated to a single MAb through a single point of attachment. To achieve this, the nano-device needs to have a single potential reaction site and be prepared through procedures that allow control over each aspect of the synthesis.

Inorganic nanodevices: Significant progress has been made in the areas of tailoring inorganic materials for biological applications. One particularly promising technology was developed by Chad Mirkin at Northwestern University. (Mirkin, C. A. et. al., Chem. Rev., 2005, 105, 1547-1562; and U.S. patent application Ser. No. 11/050,983 published as 20060068378) Mirkin's group modifies inorganic nano-scale materials with DNA for sensing and imaging applications. Again, like SCKs, this technology can not be easily adapted to prepare the desired nano-composite structure, since it is not a simple task to meet the single reaction site limitation desired in the application. Furthermore, it would be challenging to engineer all of the various desired product specifications, detailed below, into a device using an inorganic substrate approach.

Dendrimers: Dendrimer technology does present the opportunity for fabrication of single reaction site nano-devices. Much work has been carried out in J. M. J. Frechet's lab at UC Berkeley on dendrimers. (Fréchet, J. M. J. et. al., Nature Biotech. 2005, 23, 1517-26 and PNAS.0607705103) Conceptually, dendimers are an excellent choice for developing complex multi functional nano-devices, however, facile fabrication of dendrimers is not yet commercially practical. For instance, building a dendrimer with all of the features of the nanodevice described in this proposal would take, conservatively, 50 separate sequential reactions and controlled degradation, notification of identification of cancer cells, and controlled delivery of payload would not be incorporated into a dendrimer based nano-device. Furthermore dendrimers adopt a somewhat fortuitous morphology in solution, one whose shape is sensitive to changes in the solvating power of the selected solvent or in-vivo environment, therefore, they do not offer a well defined stable encapsulating environment as disclosed herein.

In summary all these current forefront approaches to novel nano-scale imaging and drug delivery devices reviewed by Ferrari and later by Chari have their limitations and a universal nano-device drug delivery platform for monoclonal antibody based therapeutics is still needed.

### BRIEF DESCRIPTION OF THE DRAWING FIGURES

While not intending to be limiting, the following figures assist in clarifying the concepts relating to the fabrication and incorporation of a desired spectrum of different functionalities into the nano-device conjugate.

FIG. 1: Simplified schematic of the structure (A) and function (B) of an exemplary multifunctional nano-device drug delivery platform for monoclonal antibody based therapeutics.

FIG. 2: Synthetic scheme for stepwise fabrication of an exemplary nano-device drug delivery platform for conjugation to selected MAbs.

### DESCRIPTION OF EMBODIMENTS OF THE INVENTION

As disclosed herein, it is possible to design such a multi-functional nano-scale device that can be conjugated to a single monoclonal antibody. An approach to design the device to deliver a multi-molecule therapeutic payload to a targeted tumor site, and further once inside a cancerous environment the shell of the device is progressively cleaved and the core of the device can adhere to the cancer cells, accumulate at the cancer site and then, and only then, release its payload of anti-cancer drugs is disclosed.

The synthesis scheme for this device is less labor intensive than a dendrimer approach and each of the polymer segments can be modified by use of one or more commercially available, low cost, monomers during the synthesis of the device thereby modifying the properties. The underlying chemistry which enables the production of the nano-device is a controlled or living polymerization process, preferably a controlled radical polymerization process (CRP), such as atom transfer radical polymerization (ATRP), nitroxide mediated polymerization (NMP) or reversible addition fragmentation transfer (RAFT). ATRP has already proven to be a very versatile, verifiable, reproducible and scalable approach for the preparation of well defined molecular structures under viable easily attained reaction conditions.

Review articles on each of these CRP procedures have recently been published. Braunecker, W. A.; Matyjaszewski, K. Progress in Polymer Science 2008, 33, 165. Sciannamea, V.; Jerome, R.; Detrembleur, C. Chem. Rev. 2008, 108, 1104-1126. Moad, G.; Rizzardo, E.; Thang, S. H. Polymer 2008, 49, 1079-1131. York, A. W.; Kirkland, S. E.; McCormick, C. L. Adv. Drug Delivery Rev. 2008, 60, 1018-1036.

One of the most critical challenges when employing polymeric materials for drug delivery in the body is avoiding damage to the kidneys. Conventional polymer synthesis results in a preparation of polymers with a wide range of molecular weights, in other words, broad molecular weight distribution (MWD). Consequently, even when very low molecular weight (MW) materials are targeted, there is a significant fraction of high molecular weight polymers formed in the synthesis. High MW polymers pose a problem for the body's renal system. (Blainey, J. D. Curr. Probl. Clin. Biochem. 1968, 2, 85.) Polymeric materials which are larger than the renal cut-off may stay in the body for prolonged periods of time and may be dangerous.

All of the polymers prepared in an ATRP synthesis have essentially the same pre-determinable molecular weight and, if desired, high MW polymers are absent. The number average MW is determined by the ratio of consumed monomer (M) to initiator. With a mono-functional alkyl halide initiator (RX), and measured monomer conversion according to Mₙ=conv×MW(M)×[M]₀/[RX]₀. Therefore polymers with a particular size, or dimension, can be made with high fidelity and excellent reproducibility.

In an ATRP all polymer chains grow at essentially the same rate, assuming fast initiation, and site specific functionality can be introduced to a molecule. PCT/US04/09905, which is hereby incorporated by reference, describes the preparation of high MW polymers with bio-degradable units evenly dispersed along the polymer backbone thereby confirming that higher MW polymers can be prepared with regularly distributed degradable functionality to circumvent the renal threshold problem.

ATRP is a living/controlled polymerization process and each polymer chain retains ω-terminal functionality and can be chain extended or functionalized. This technology has made it possible for researchers to create a variety of previously unattainable complex nanostructures i.e. brushes, mikto aim stars, and grafted surfaces.

One aspect of the exemplary device described below that demonstrates the synthetic versatility of the described procedure for the nano-device and utility of the nano-device is that the residual α-terminal functionality present on the device can be designed to be conjugated to lysine truncation variants on the Fc of a MAb. (Froidevaux, et al. J Nucl Med. 2004, 45, 116-123.) Since the Fc region is not variable, most FDA approved MAbs and MAb-based therapeutics in the pipeline will be compatible with this device and the tethered nano-device will not modify the structure, tolopogy and selectivity of the MAb.

Although we exemplify the device's potential by focusing on the preparation of a nano-device that can be conjugated to a specific MAb that will be selected to recognize a particular cancer cell line and hence deliver a specific loading or combination of anti-cancer drug(s) to a specific type of cancer, the versatility of the underlying chemistry will allow the device to be designed to recognize and address many delivery/identification/tumor/imaging/drug delivery systems.

A broader spectrum of MAb's can be employed to target all aspects of the cancer environment. In the specific field of drug delivery the nano-device can be designed to have the capacity to deliver more than four, indeed more than ten or twenty or more anti-cancer agents per nano-device, indeed up to 100's of individual drug molecules if desired, it will have significantly increased potency over direct small molecule conjugated MAb's. This increase in potency will reduce patient side effects from immunogenic response caused by MAb aggregation because it will allow biopharmaceutical companies to make formulations with higher concentration of encapsulated toxic drugs and with lower concentrations of MAb per treatment.

Of particular relevance to the disclosed invention of a multi-functional nano-device conjugate, which will be specifically addressed herein without limiting the broad applicability of the universal drug delivery platform for monoclonal antibody based therapeutics disclosed herein, is design of a nano-device at the molecular level for encapsulation of anti-cancer drugs that further provides a means for conjugating anti-cancer agents to MAb's thereby delivering highly toxic agents directly to cancerous areas, while sparing normal tissue from exposure to toxic drugs and reducing the stress of chemotherapy.

There has been some recent research which aims at conjugating small molecule cancer agents directly to MAb's. [Chari, R. V. J. Acc. Chem. Res. 2008, 41, 98-107.] Small molecules like radioisotopes, bacterial toxins and small-molecule drugs have been linked to antibodies through peptides, protein fusions and chelating agents. [Allen, B. J. Rev. Recent Clin. Trials 2008, 3, 185-191. Kelly, M. P.; et.al; Cancer Biother. Radiopharm. 2008, 23, 411-424.] However, as noted above, only a few drug molecules are delivered by each conjugate.

The disclosed device is designed to deliver larger amounts of identifying, imaging or therapeutic agents per bio-recognition event. This pre-determinable, pre-selectable spectrum of functionality is the fundamental advantage of the disclosed nano-vector with primary MAb functionality, or addressing functionality, selected to include avoidance of bio-barriers through biomarker-based targeting while the bio-active imaging or therapeutic agents are encapsulated within the core of the device.

The disclosed nano-device conjugate could be considered to be a polymer and while a wide variety of polymeric materials are currently being used as implants, coatings for implants and as drug delivery devices (Ali, M.; Brocchini, S. Advanced Drug Delivery Reviews 2006, 58, 1671-1687; Burke, S. E.; Kuntz, R. E.; Schwartz, L. B. Advanced Drug Delivery Reviews 2006, 58, 437-446; Schwarz, M. C.; Richard, R. E.; Zhong, S.-P. In PCT Int. Appl.; WO/2005113031, 2005, 26 pp) successful utilization of polymeric materials within the body still presents challenges. As noted above, one of the most critical challenges is avoiding damage to the kidneys. Conventional polymer synthesis results in a preparation of a population of polymers with a wide range of molecular weights, in other words, broad molecular weight distribution (MWD). Consequently, even when very low molecular weight (MW) materials are targeted, there is a significant fraction of high molecular weight polymers formed in the synthesis. High MW polymers pose a problem for the body's renal system. Polymeric materials which are slightly larger than the renal cut-off, get stuck in the renal system, clog the organ, and can lead to death of the patient. Living/controlled ionic and radical based polymerization processes circumvent this problem by preparing polymers with preselected molecular weight and narrow MWD and site specific degradable linking groups between polymer segments.

ATRP technology is one such controlled/living polymerization process and is used herein, as one of many such controlled polymerization processes known in the art, as an exemplary process that allows the scientist to make polymers with predetermined controlled MW, very narrow MWD and site specific functionality. Reversible addition fragmentation transfer, (RAFT) (McCormick, et.al, Polymer Reviews, 2006, 46, 421-443; Moad, G.; et.al.; Macromolecular Symposia 2003, 192, 1-12) is another controlled radical polymerization (CRP) process and in one embodiment is a preferred CRP process as the transfer agents can be converted to thio-groups that can be employed as degradable linkages for tethered drugs. All of the polymers, or polymer segments, prepared in a “living”/controlled polymerization process can have essentially the same molecular weight. High molecular weight polymers are absent from the resulting synthetic mixture. In addition, CRP allows the researcher to pre-determine the molecular weight by adjusting the reaction conditions. Polymers with a particular size or dimension can be made with high fidelity and excellent reproducibility.

The invention and development of ATRP technology, by Kris Matyjaszewski at Carnegie Mellon University, has been disclosed in a series of papers (J. S. Wang, K. Matyjaszewski, J. Am. Chem. Soc., 117, 5614, 1995; Matyjaszewski, K.; Xia, J. Chem. Rev. 2001, 101, 2921-2990 and in a series of patents and patent applications with Matyjaszewski as inventor and Carnegie Mellon University as assignee, (U.S. Pat. Nos. 7,125,938; 7,064,166; 7,056,455; 7,049,373; 7,019,082; 6,887,962; 6,790,919; 6,759,491; 6,627,314; 6,624,263; 6,624,262; 6,541,580; 6,538,091; 6,512,060; 6,407,187; 6,162,882; 6,124,411; 6,121,371; 6,111,022; 5,945,491; 5,807,937; 5,789,487; 5,763,548 and their international equivalents) all of which are hereby incorporated by reference to define the scope of ATRP. ATRP has made possible the design and fabrication of complex nano-scale structures. As described above, ATRP allows preparation of polymers with well defined, pre-determined molecular weight. Further, ATRP is a living radical polymerization process, which means that any radically (co)polymerizable monomer can be utilized in the direct synthesis of the polymer and that the first polymers generated by ATRP reaction can be put back into the reaction flask and chain extended or the reactive end group(s) can be funtionalized using known chemistry. Further polymers prepared by other procedures can be modified with additional initiator(s) and chain extended. This technology has made it possible for researchers to create a variety of previously unattainable complex nanostructure i.e. brushes, [Tsarevsky, N. V.; Bencherif, S. A.; Matyjaszewski, K. Macromolecules 2007, 40, 4439-4445.] mikto arm stars, [Gao, H. and K. Matyjaszewski (2008) Macromolecules 41(12): 4250-4257.] and grafted surfaces [McCarthy, P., et al. (2006) ACS Symposium Series 944: 252-268.].

Indeed, successful ATRP of the monomers employed in the initial exemplary examples below including polyethylene glycol (PEG) methacrylate, dimethylamino)ethyl methacrylate, (DMAEMA) and its alkylated analogues, butyl methacrylate, and t-butyl methacrylate are well documented in incorporated references including J. Am. Chem. Soc. 2006, 128, 5578 and Macromolecules 2004, 37, 9768 and references therein. Not only homopolymers but also random, gradient, and block copolymers derived from these monomers have been described. The synthesis of molecules with complex architectures, including brush-like molecules, has also been reported. However prior art papers and patents describing the preparation of brush copolymers have focused on attaining high molecular weight polymers with well defined observable structures and not on preparing materials with lower molecular weight segments that incorporate multiple designed functionality.

The synthesis of brush macromolecules usually involves the initial synthesis of a hydroxy-group containing backbone, which can then be converted to a multifunctional macroinitiator by a simple esterification reaction with a 2-bromocarboxylic acid. Grafting from the backbone is the next step, and numerous examples of high molecular weight bottle brush polymers with homopolymer, random, gradient or block copolymer backbones have been reported.

CRP processes, including ATRP, are very powerful techniques for the preparation of functional polymers. Four strategies (Scheme 1) have been utilized for the introduction of one or more functional groups:
i) direct polymerization of a functional monomer,
ii) polymerization of monomers with protected functional groups that are released after the polymerization,
iii) use of functional initiators, (an initiator incorporating a bioresponsive functional group was disclosed by Maynard in J. Am. Chem. Soc. 2004, 126, 15372-15373) and post polymerization chemistry on the end-group. (ACS Symp. Ser. 2006, 937, 79.)

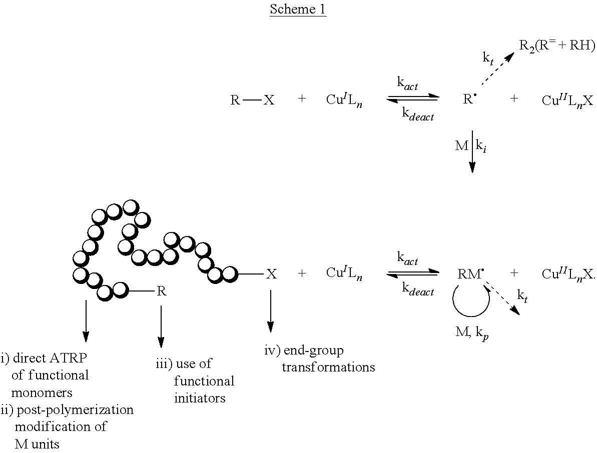

The introduction of a hydrolytically degradable ester group in a polymer backbone has been achieved by the ATRP of (meth)acrylates in the presence of a monomer with an exocyclic double bond, 5,6-benzo-2-methylene-1,3-dioxepane (BMDO), (Polymer 2005, 46, 11698.) whose reaction with radicals leads to ring opening and formation of ester group in the backbone of the copolymer. Various degradable polymers (linear, hyperbranched, star-like, crosslinked (U.S. Application 60/850,453)) with one or more disulfide groups have also been prepared by ATRP.

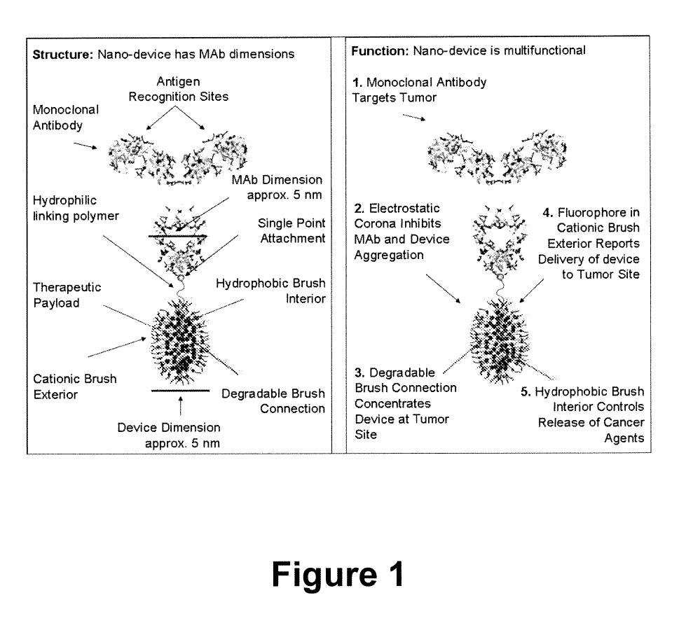

These established protocols will be used in the syntheses of the disclosed novel monofunctional nano-device drug delivery platform designed to address the present problems with MAb based treatments. A schematic of the structure displaying some of the predetermined functions of the device is shown in FIG. 1.

The target nano-device platform has multiple functions including:
First, the nano-device will have site specific targeted delivery through conjugation to a selected monoclonal antibody.
Second, the device is designed to reduce the side effects of present MAb systems.
Third, the nano-device will concentrate delivery of its payload at the targeted site, (in the figure cancer is targeted).
Fourth, the nano-device will release the payload (exemplified herein by an anti cancer drug) at a controlled rate.
Fifth, optionally, the nano-device will report that the device has been delivered to the tumor site.
Sixth, optionally, the nano-device will comprise an imaging agent that will allow the site of the targeted tumor to be identified.

As noted above the conjugated nano-device can be constructed to target a number of disease sites within the body by selecting the appropriate MAb for conjugation to the nano-device. Suitable MAb's include for example MAb's developed by Genentech, Protein Design Labs, Human Genome Sciences, Biogen IDEC, and Immunogen.

In one embodiment nanodevices encapsulating different fluorophore, or fluorescent molecule, can be conjugated to specific MAb's to notify delivery of the device to a different specific tumor site. Indeed this phenomenon of targeted delivery of a specific fluorophore to different targeted cancers could be employed in a preventative care environment to determine if a patient has cancer in the first place, what type of cancer should be treated and if the fluorophore is replaced by an imaging molecule where the cancer is situated.

In the following non-limiting discussion of the utility of the device, the exemplary discussion will focus on eradication of cancer tumors as the targeted application.

Function 1: Device Targets Tumor(s). We have designed this device to have a single functional site that can be conjugated to the Fc of a MAb. We chose to do this by employing a functional initiator that comprises a functional group that can be utilized for device linkage chemistry specific for lysine truncation variants on the Fc of a MAb. Since the Fc region is not variable, most FDA approved MAb's, and MAb-based therapeutics in the pipeline, will be compatible with this device. In this sense, the device is universal and is designed to attach to a single site on the MAb. The specific MAb that is selected to be conjugated to the device will define which type of disease, and in the non-limiting exemplary case discussed herein, which specific type of cancer the device will target and thereby deliver the device and its multi-molecule drug load to the selected target tumor site(s). Since the nano-device will have the capacity to deliver from a single digit number of molecules to hundreds of anti-cancer agents per device, it will provide a significant increase in potency over conjugation of a small number of drug molecules directly to the MAb's.

This increase in potency will reduce patient side effects from immunogenic response caused by MAb aggregation because it will allow biopharmaceutical companies to make deliverable formulations containing the required concentration of the desired drug(s) with lower concentration of the MAb.

This reduced MAb concentration also will reduce the extent of MAb aggregation and make the drug easier to administer.

Furthermore, by changing the composition of the core of the nano-device, to increase the chemical affinity of the core to match the phylicity of the drug(s) desired to be encapsulated, the device will be able to be encapsulate almost any drug and further, by selecting the first non-initiating functional group on the initiator, the device can be designed to be conjugated to any biomolecule or substance.

The chemical affinity of the drug to the core can be increased by incorporation of second functional groups into monomers used in the core forming (co)polymerization that interact with the encapsulated molecules by various non-chemical bond forming reactions such as a combination of van der Waals interactions between host and guests and differential internal strain and electrostatic interactions including ionic interactions, non-covalent hydrogen bonding interactions, non-covalent directed interactions formed via molecular imprinting techniques, ligand-receptor interactions, interactions via protonated amine groups and the negative or high electron density fragments of nucleosides or nucleotides etc.

In an exemplary example which specific MAb is conjugated to the device will define to which type of cancer will be targeted. Since the nano-device will have the capacity to deliver more than five or ten, indeed more than twenty, and if desired even hundreds of anti-cancer agents per MAb linked nano-device, it will have significantly increased potency over small molecule drugs directly conjugated to MAb's. In addition the length of the link between the MAb and the nanodevice can be sufficiently long to allow the MAb complete conformational freedom to enhance its ability to target the cancer. This increase in potency will reduce patient side effects from immunogenic response caused by MAb aggregation because it will allow biopharmaceutical companies to make formulations with lower absolute concentrations of MAb.

Function 2: Device Reduces MAb Treatment Side Effects. In addition to increasing the potency of the MAb conjugated drug delivery devices by carrying more than a few directly linked drug molecules to the targeted site, the device can be designed to reduce MAb aggregation since the peripheral shell of device can optionally incorporate functionality that can reduce, or even inhibit, physico- and chemico-MAb aggregation. Therapeutic MAbs are basic proteins. The isoelectric point (pI) of humanized therapeutic MAbs range from pI=8 to pI=10. If the pH of the solution is less than the pI of the MAb, then the MAb has an overall positive charge, indeed most MAb's have a net positive charge in physiological environments. Knowing this, the device can additionally be engineered to have a net positive hydrophilic shell. This additional adjustable positive charge will reduce the probability of intra- and inter-molecular conjugated device collapse in addition to reducing nano-device aggregation.

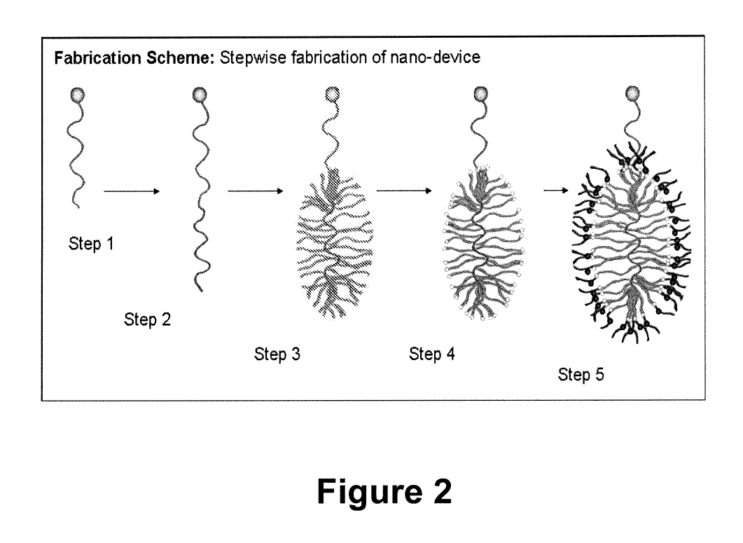

One embodiment that can be employed for the synthesis of the nano-device that will be conjugated to the selected MAb or fragment thereof is shown schematically in FIG. 2 and described in detail below. If the nano-device is constructed in this manner, the device and the conjugated MAb possess the same charge and thus electrostatically repel each other. This mutual electrostatic repulsion will not only keep the device from collapsing onto the MAb, intra-molecular collapse, but it will keep other MAb's from interacting with both the device and its host MAb, inter-molecular collapse. This feature of the device will inhibit, or reduce MAb aggregation by increasing the repulsive forces between MAb-device conjugates thereby decreasing the level of the immunogenic response within the patient's body when a foreign substance is detected.

As noted above, early research in drug conjugated MAb's used direct drug-MAb conjugations that could be cleaved by pH dependent mechanisms. However, additional research determined that too much of the drug was being released in the blood and that a cleavable link based solely on pH was too promiscuous. In one form of the designed nano-device disclosed herein there are a multiplicity of pH responsive linking units between the device core and the device shell. If some of the links are degraded during transportation the remaining links preserve a hydrophilic shell around the device and no drugs are released from the hydrophobic core. Furthermore, the degradable links can also be designed to be cleaved by an enzyme inside the targeted cell whose activity is pH dependant, thereby reducing the probability of the drug being release into a patient's bloodstream.

Function 3: Device Concentrates Payload at the Tumor Site. The potency of site specific MAb's will also be increased because the device is designed to recognize, (via selection of appropriate MAb's), the targeted cancer and hence concentrate itself at the targeted tumor site(s) after it has been delivered into the body. A further functionality can be engineered into the device to enhance the efficiency of drug delivery. This is accomplished by incorporating an environmentally responsive biodegradable unit between the hydrophilic and hydrophobic blocks of the device. In this embodiment of the nano-device delivery system the hydrophilic exterior block, or shell of the nano-device, which is responsible for inhibiting MAb aggregation, intramolecular device collapse and device precipitation during transportation through the body will be tuned to completely degrade only in cancerous environments. The shell of the device completely cleaves from the drug encapsulating core only in cancerous environments. Once inside a cancerous environment, each linker will cleave and release each individual hydrophilic exterior tethered chain segment of the device at the targeted site. Cleavage can occur by exploiting slight differences between the environment of a cancerous tumor and a normal cell including differences in pH, presence of different enzymes, and differences in the level of oxygen present in the rapidly expanding cancerous tumor.

The interior of the device, which is designed to be loaded with the drug, is hydrophobic. Once the hydrophobic interior, or core of the nano-device, is exposed, this part of the device is no longer soluble or dispersible in aqueous environments and precipitates out of solution, sticks to the tumor site through hydrophobic interactions, and thus the payload of several MAb conjugates can be concentrated at the tumor site.

A further advantage of encapsulation of the drugs within the core of the nanodevice is that encapsulation shields the anti-cancer agents from the in vivo environment which allows the user to load inherently unstable anticancer agents within the device. The nano-device will protect the agents from degradation during transportation and deliver the active agent to the targeted site.

Function 4: Device Controls Rate of Drug Delivery to the Tumor. Once the device has concentrated at the tumor site the anti-cancer drug(s) elute from the device. The rate at which the drug(s) elute is tunable by modifying the hydrophobicity, or drug compatibility, of the device interior of the nano-device and the rate of removal of the hydrophilic shell.

In one embodiment of the invention the interior of the device comprises a gradient copolymer which can be designed to result in an initial burst of drug followed by a more prolonged release of the residual drug(s) to ensure killing of all the cancerous cells in the nearby environment.

In another embodiment the hydrophobic interior of the nano-device comprises additional functionality which can form a degradable link with a fraction of the encapsulated drug to control the rate of elution.

In another embodiment the rate of drug elution is further modified, or controlled, by controlling the rate of cleavage of some fraction of the hydrophilic shell. The slower cleaved shell fragments act to reduce the porosity of the core and hence the rate of diffusion of the drug from the core.

One procedure that can be employed to adjust the free volume within the core of the nano-device is preparation of a copolymer during step 2 of scheme 2. In this embodiment the functional monomer that additionally comprises an ATRP initiation functionality, or a precursor thereof, is copolymerized with a monomer that does not comprise the functional unit employed to initiate an ATRP to form the tethered core forming chains in step 3. In a good solvent for the core a greater free volume is created by incorporation of a fraction of non-grafted backbone units allowing additional drug molecules to be incorporated or encapsulated by the core of the nano-device.

Another tool to increase the free volume within the core is to tether a longer core chain, step 3 FIG. 2, to the first backbone molecule.

Both procedures can be employed simultaneously.

Function 5: Device Reports Delivery to the Tumor. In one embodiment of the invention the segment of the tethered block polymer segments which comprises the hydrophilic layer or shell of the nano-device will be copolymerized in the presence of a small amount of a monomer containing a fluorophore, such as pyrenemethyl methacrylate, or some other visibly or spectroscopically detectable unit, can be added to the polymerization and thus, incorporated into the brush. When the hydrophilic exterior is released at the tumor site, it will take with it the incorporated detectable unit and, since this polymer segment containing the fluorphore, or other detectible unit, comprises a molecular weight below the renal threshold it will be detectable in urine samples.

In a further embodiment of the invention this urine detectable unit is the only functionality incorporated into nano-device conjugate and when the nano-device comprising different fluorphore units is conjugated to a series of MAb's targeting specific cancers they can be used to detect specific cancerous environments within the body and provide information to the patient/doctor that one or more particular type(s) of cancer is present and further diagnosis/treatment is required. In this embodiment several different devices can be conjugated to several different MAb's to detect the presence of several different types of cancer. The particular fluorphore unit initially detected will identify which cancers are present. Such a series of nano-device conjugated MAb's can be employed in an annual screening test to identify early stage cancers.

Function 6: Image enhancement. In a further embodiment of the invention an additional non-drug encapsulated image enhancing functionality is incorporated into the core of the nano-device conjugate and when such nano-devices conjugated to a series of MAb's detects specific cancerous environments within the body and the urine detectable unit informs the patient/doctor that cancer is present the further functionality, such as encapsulated metallic nano-dots or other image enhancers, exemplified herein by incorporating within the core a chelating site chain which allows loading the nano-device with gadolinium (Gd) which is clearly seen in subsequent MRI screening tests.

Device Construction: The device is preferentially constructed using controlled polymerization procedures. ATRP, RAFT and nitroxide mediated polymerization (NMP) are suitable controlled radical polymerization processes (CRP). The dimensions of a (co)polymer prepared by a CRP can be controlled at the nanometer level. Furthermore the formed molecule can possess predetermined, site specific, functionality.

In a non-limiting example each of the dimensions of the nano-device can be individually controlled and each can individually be less than fifty, or indeed less than twenty, or even less than ten nanometers and the device will conjugate to the MAb through a single point of attachment.

As shown in FIG. 1 the nano-device is essentially composed of a block copolymer wherein one block is a bottle brush block copolymer which has a hydrophobic interior which is shielded from the in vivo environment by a hydrophilic exterior with an attached fluorophore, or other photoresponsive unit, reporter. The hydrophilic brush exterior and the hydrophobic brush interior are linked by a unit which can be selectively cleaved or degraded in a cancer environment. This will result in release of the hydrophilic segments, optionally resulting in detection of the hydrophilic segments in urine samples, and exposure and in situ precipitation of the hydrophobic brush interior within the tumor, which results in an agglomeration of the core of numerous devices within the cancer tumors or cancer cells. Independent of the hydrophilic brush exterior, the drug(s) are bound by chemical affinity, or in various embodiments attached to, the hydrophobic brush interior and releases only within a cancer environment at a rate controlled by the composition and architectural construction of the hydrophobic polymer interior of the nanodevice.

Suitable degradable linking units include disulfides. In one exemplary example these units can be incorporated by conversion of the first terminal Br unit present in a typical ATRP to NH₂ and then reacting it with maleimide-N-spacer-S2-spacer-OCOC(Br)Me₂ thereby providing a second initiating Br functionality beyond the degradable unit for a second ATRP of the hydrophilic shell.

In another embodiment the —OCOC(Br)Me₂ group could be replaced by a preformed biocompatible polymer, such as preformed PEO-chains, or proteins.

In another embodiment a RAFT process could be employed for the preparation of the core of the nano-device and the transfer agent replaced by interaction with a thio-terminated hydrophilic polymer which can comprise synthetic polymers such as PEO- or natural bio-polymers such as proteins.

In a sequential ATRP process for the preparation of the nano-device there are several strategies to introduce the disulfide group-containing ATRP initiator, one synthetic strategy is shown below in Scheme 2. The mixed disulfides can be synthesized using standard procedures. The nature of the groups Y will be selected to meet the requirements regarding the targeted rate of degradation at certain pH values in specific cancerous environments, both alone and in the presence of enzymes.

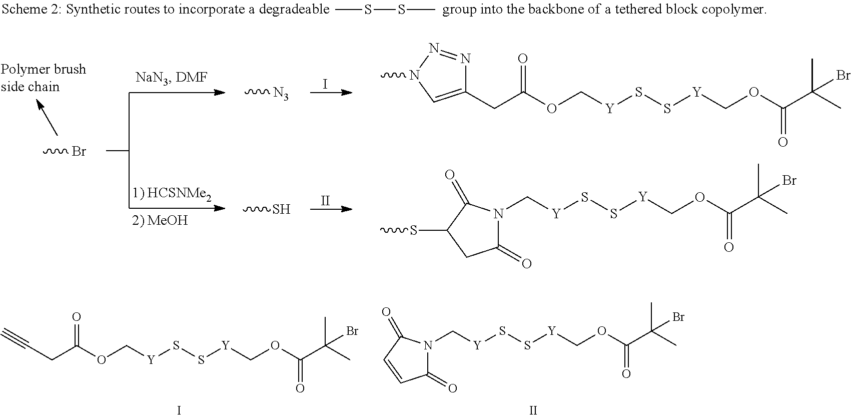

The efficient replacement of alkyl bromide end groups in a series of polymers with both azide and thiol functionality has been demonstrated. (Matyjaszewski, K.; Nakagawa, Y.; Gaynor, S. G. Macromol. Rapid Commun. 1997, 18, 1057-1066; Tsarevsky, N. V.; Matyjaszewski, K. Macromolecules 2002, 35, 9009-9014.) This allows incorporation of functionality into the chain end of our nano-device that will be utilized to directly conjugate the nano-device to the C-terminal lysine residue of the MAb.

Several biopharmaceutical companies including; Genentech, Protein Design Labs, Human Genome Sciences, Biogen IDEC, and Immunogen are preparing MAb-drug-tumor combinations and each and every one of these MAb materials can be conjugated to the nano-device thereby allowing the nano-device to target specific cancers or other bio-environments. Conjugation of the MAb's to the nano-device will provide the selected MAb with the added advantage that multiple drug molecules can be remotely attached to the specific MAb through a single linkage and delivered to the targeted site with little or no drug leakage from the designed nano-device and furthermore with reduced aggregation of the MAb's thereby improving the efficiency of each treatment.

Several pharmaceutical companies including; Amgen Inc., Affymetrix Inc., Protein Design Labs Inc, Pro Pharmaceuticals Inc, Threshold Pharmaceuticals Inc, Tanox, Inc., are preparing anti cancer drugs that can be encapsulated within the core on the nano-device for delivery to targeted specific cancers. Furthermore, because the drugs are encapsulated within a device designed to trap all drug molecules within the core of the device, drugs that are too aggressive for present delivery systems can be employed; thereby increasing the efficiency of the treatment.

A simplistic view of the role of the nano-device, or nano-vector, is that represented by an “envelope”. An envelope that can be filled with the selected drug, sealed, then a specific address, or addresses (selected by conjugating the device to one or more of the desired MAb's) is attached to the device ensuring delivery of the envelope to the specific address, whereupon the envelope opens itself and delivers the drugs predominately to the selected address at the desired rate with little impact on the neighborhood.

Device fabrication occurs in several steps (FIG. 2) and control can be exercised over the composition, molecular weight and functionality of each polymer segment constructed at each fabrication step.

The polymers prepared in each step are characterized by GPC, NMR, etc. to ensure that the individual building blocks of the device meet the desired stringent device size and compositions specifications.

There are many degradable functional groups that can be inserted between the first attached hydrophobic block and the second hydrophilic block (step 4 in FIG. 2). They can be selected to control the rate of device degradation in the cancerous environment. The functionality of the linking group can be selected to modify the fundamental parameters of the bottle brush block copolymer to optimize the rate of degradation so that the device will stick to the cancer cells even after the linking group between the device and the MAb has degraded or the MAb itself has degraded.

The composition of the backbone segment of the nano-device (formed in step 2 of FIG. 2) can be controlled to adjust the free volume of the inner hydrophobic core so that differing concentrations of drugs or individual number of drug molecules can be loaded into the core and furthermore can be eluted from the precipitated core fragments at a desired rate.

In another embodiment of the device the initiator functionality for growth of the core chains in step 3 are attached to the brush-backbone segment through a degradable link, such as an ester, to allow slow degradation of the agglomerated core units after the drugs have been delivered. The MW of the degraded core units will be below the renal threshold.

As noted above the nano-device can be conjugated to a spectrum of model MAb molecules through a single functional unit and characterized to determine the quality of conjugation and ability of the conjugate to migrate to, identify, and destroy different cancer cells.

The synthesis of the nano-device can be considered to be accomplished by a series of exemplifying but non-limiting series of well controlled ATRP polymerization steps, see FIG. 2.

In step 1: since it is desired that the nano-device be capable of conjugation to the MAb delivery system, a functional initiator for an ATRP polymerization of a biocompatible monomer, such as a PEG-methacrylate monomer or 2-(methacryloyloxy)ethyl phosphorylcholine, or a preformed bio-compatible macromonomer, is selected that has an additional functionality for subsequent conjugation to a MAb. The residual functionality on the initiator is chosen such that known efficient chemistry allows this chain end to conjugate with the Fc of the MAb or, is selected to be easily converted to a functionality that can be efficiently conjugated with the MAb.

The synthesis conditions for the linking segment between the nano-device and the MAb conjugate, step 1, can be adjusted to produce a polymer whose degree of polymerization can be controlled at the nanometer level. E.g if this segment of the nano-device is desired to be 2 nm long then a degree of polymerization (DP)=7 would be targeted, (although any range of segment lengths could be constructed and chain end functionality would be available for linking as long as the molecular weight was below the chain entanglement molecular weight of the first copolymer segment.) Indeed any targeted MW below DP=50, is desired.

Furthermore, the diameter of the linking chain formed in step 1 can be controlled. If the diameter was targeted to be 2 nm wide this could be predetermined by consideration of the molecular weight of the PEG side chains on the methacrylate (macro)monomer. As noted above with the length of the linking segment formed in step 1, this can also be varied as desired by selecting the appropriate oligo/macromonomer for polymerization in this first step.

The material prepared in this initial step is chain extended with a monomer bearing a functionality that is, or can be converted into, an ATRP initiating functionality. A wide range of suitable monomers have been disclosed in incorporated patents and in several papers discussing the synthesis of bottle-brush molecules. An exemplary functional monomer, first disclosed in U.S. Pat. No. 6,541,580 and described in Macromolecules 1998, 31, 9413-9415 which is also incorporated by reference, is HEMA-TMS.

Step 2 in FIG. 2: the synthesis conditions for this second step can also be adjusted to produce a poly(co)(HEMA-TMS) unit which is of predetermined length, e.g. if the desired length is 5 nm long then a DP=14 would be targeted. A result with average DP ranging from 10 to 20 and PDI less than PDI=1.3 would be acceptable. This is scaleable in that a targeted DP of 30 would result in a 10 nm segment with a PDI lower than 1.3.

HEMA-TMS units within the block copolymer from step 2 will be converted to HEMA, then the —OH group of each HEMA unit will be converted to an ATRP initiator through a simple esterification reaction as disclosed in incorporated patent applications and references. HEMA, or other hydroxyl containing monomers can be directly copolymerized in step 2 if desired. (Beers, K. L.; Boo, S.; Gaynor, S. G.; Matyjaszewski, K. Macromolecules 1999, 32, 5772-5776.)

Step 3 of FIG. 2: the hydrophobic polymer segments that will form the core of the nano-device are formed by (co)polymerization of hydrophobic monomer(s), such as an alkyl (meth)acrylate, from the multifunctional block copolymer formed in step 2.

The free volume of this brush block copolymer in a good solvent can be measured to provide an indication of the drug carrying capacity of the final nano-device.

In one embodiment, if a higher capacity is desired then this can be addressed in step 2 by increasing the MW of the polymer segment prepared in step 2; or in another embodiment by conducting a copolymerization with a non-functional monomer to incorporate non-initiating monomeric species into the second polymer segment, thereby increasing the spacing of the final grafted chains along the backbone segment formed in step 2, thereby increasing the free volume of the core of the nano-device in a good solvent. In a non-limiting example, presently being employed for exemplification of a method to increase the free volume of the core, this comprises copolymerization of HEMA-TMS with a non-functional monomer which can increase available free volume without significantly increasing brush dimensions.

In a further embodiment the drug carrying capacity of the nano-device can be addressed in step 3 by (co)polymerizing a monomer comprising a less bulky alkyl-group. Or by preparing a tethered (co)polymer chain of higher MW. The particle size of this initial nano-device in a good solvent and a poor solvent will thereby indicate the free volume available within the first formed hydrophobic brush copolymer for drug encapsulation.

Again, as discussed above an acceptable result when targeting a device with a 5 nm long core segment could be an average DP of the first tethered core segment ranging from 6 to 10 and PDI less than PDI=1.3 but, as in all controlled polymerization processes, the dimensions of the core forming segments can be adjusted at will by one skilled in the art by varying the molar fraction of initiating species to consumed monomer(s) and degree of conversion.

In step 4 of FIG. 2: a degradable linking unit is incorporated into the active tethered polymer segments by modifying the chain end of the hydrophobic core. Exemplary degradable linking groups have been disclosed in WO 2004087777.

In one embodiment of the invention the degradable linking groups can comprise a disulphide containing initiator. A non-limiting set of disulphide degradable linking groups can be selected from a group of structures which have previously been identified to degrade only within cancer environments. The structure of the degradable linker unit can comprise a “spacer” in the structure which can be aromatic or aliphatic; both may optionally comprise additional functionality to modify the rate of degradation. When an aliphatic unit is selected, one with an alpha-substituent next to the sulfur, that is an electron-withdrawing or a donating group, (such as —CN, —COOR, and —OR) the selected group affects the rate of degradation in different cancerous environments.

In step 5 of FIG. 2: the precursor of the hydrophilic shell exemplified by an anionic polymer further exemplified herein by poly(trimethylammonium ethylmethacrylate), poly(TMAEMA) or poly(DMAEMA) based segments (again the DP of the segment can be selected to=5, or if a larger device is targeted >5) will be grown from the terminal initiators to form the brush macromolecule comprising a di-block “bristle” with a degradable unit between polymer segments of different phobicity.

If it is desired that the degradation of the nano-device be monitored then an imaging (co)monomer, such as one comprising a fluorophore is incorporated into this segment of the nano-device. In an exemplary example a result with average DP ranging from 3 to 7 and PDI less than PDI=1.3 would be acceptable for a nano-device targeting 10 nm in overall dimensions.

The final dimensions of the nano-device can be measured by light scattering in solution or by AFM after depositing a solution of the device on a mica substrate.

Optimization of drug elution. The nano-device prototypes can be loaded with the desired anti cancer drug(s), or an anti-cancer drug analog(s), and the rate of drug elution in both cancer and non-cancer environments measured by placing drug loaded devices into a dialysis tube and then measuring differences in rate of migration through the membrane when the drug has been loaded into the device and when the drug has not been loaded into the device by UV/Vis, GC or HPLC.

An acceptable result will be one in which the linkage does not degrade in an environment mimicking a healthy cell but does degrade in an environment mimicking a cancer cell.

Thereafter the nano-device prototypes will be loaded with an anti cancer drug. Doxorubicin will be used as a proof of concept anti-cancer drug.

Since, in one embodiment the nanodevice is designed to release the drug only at the targeted cancer site this exemplification can result in synthesis of a device which can be loaded with agents which, if administered alone, would be deemed to be too toxic for administration to a patient. Hydrophobic drugs will have a high affinity to the hydrophobic interior of the cylindrical polymeric brush. Rate of drug elution in both cancer and non-cancer environments will be measured.

The benefits of this novel MAb conjugated device include the fact that site specific delivery of the nanodevice to the MAb addressed site allows the user to load highly toxic anti-cancer agents within the protected core of the device. Encapsulation shields the anti-cancer agents from the in vivo environment which allows the user to optionally load inherently unstable anticancer agents within the device. The nano-device will protect the agents from degradation during transportation and deliver the active agent(s) to the MAb targeted site.

Furthermore the efficacy of the MAb conjugated device lies in the single terminal functionality on the first formed nano-device which allows conjugation of the nano-device to any existing MAb which targets any aspect of the cancer cycle.

### EXAMPLES

Homopolymer, block copolymer, and brush copolymer synthesis and characterization were carried out as in several different previous applications and publications targeting high molecular weight bottle brush macromolecules. Monomers including PEG methacrylate, TMAEMA, DMAEMA, butyl methacrylate, t-butyl methacrylate, and degradable monomers additionally incorporating ester, thiol, units have been incorporated in brush structures.

In this application prior art preparations of higher molecular weight polymers are incorporated to show the feasibility of building the targeted multifunctional nano-device.

An efficient route to evaluate the degradability of the nano-device is to study the degradation of each selected degradable link in a simple block copolymer. Therefore cleavage of the degradable linkage in a tumor mimetic environment will be monitored by GPC, as described in incorporated references. In one embodiment of the invention more than one degradable link will be incorporated into the nano-device. In a further embodiment more than one degradable link will be incorporated into the nano-device to link the core and shell polymer segments so that the shell will be removed over a longer time frame further controlling the rate of drug elution from the core.

The structure of a MAb is generally defined as having two heavy chains and two light chains connected through disulphide linkages. The V-shaped MAb also has defined regions, the Fab and the Fc. The Fab regions are the most variable in composition, but, they are also the regions that allow the MAb to recognize various antigens. The C-terminal lysine residues are in the Fc region of the MAb (the base of the Y). Our device will be designed to conjugate to the C-terminal lysine residues of the MAb in an attempt to reduce or even avoid the device from interfering with MAb antigen recognition interactions.

### Example 1

The following examples target polymers of higher molecular weight than desired for nano-device construction but are included as comparators to show the validity of the construction plan.

Materials. 2-(Trimethylsilyloxy)ethyl methacrylate (HEMA-TMS, 97.4% (GC)) was prepared as described in Macromolecules 1998; 31: 9413. Poly(ethylene glycol) methyl ether methacrylate, H₂C≡C(CH₃)COO—(CH₂CH₂O)ₙCH₃, (PEOMA, MWₐᵥ=300 g/mol, DP_{PEO}=5; MWₐᵥ=1100 g/mol, DP_{PEO}=23) were obtained from Aldrich. Antioxidants MEHQ and BHT were removed from monomers by passing through an alumina column. PEOMA with higher MW, which is solid at room temperature (rt), was dissolved in tetrahydrofuran; after removing of the inhibitor, the solvent was evaporated and the macromonomer was dried under vacuum to a constant weight. Copper(I) bromide (CuBr, Aldrich, 98%) and copper(I) chloride (CuCl, Acros, 95%) were purified by stirring with glacial acetic acid, (Fisher Scientific), followed by filtration and washing the solid three times with ethanol, and twice with diethyl ether. The solid was dried under vacuum (1×10⁻² mbar) for 2 days. Copper(II) bromide (CuBr₂, Acros, 99+%) and copper(II) chloride (CuCl₂, Aldrich, 99.99%) were used as received. p-Toluenesulfonyl chloride (TosCl, Aldrich, 99+%) and 2,2′-azobis(izobutyronitrile) (AIBN, Aldrich, 98%) were recrystallized in hexane and ethanol respectively, then filtered, and dried.

4,4′-Di(5-nonyl)-2,2′-bipyridyne (dNbpy), tris(2-ethylhexyl acrylate aminoethyl) amine (EHA₆TREN) and cumyl dithiobenzoate (CDB) were prepared as described previously. N,N,N′,N″,N″-Pentamethyldiethylenetriamine (PMDETA, Aldrich, 99%), ethyl 2-bromoisobutyrate (EtBriBu, Aldrich, 99%), all solvents and internal standards were used without further purification.

### Polymerization of PEOMA by “Grafting Through”: (Exemplary of Step 1)

PEO macromonomer, ligand (with the exception of EHA₆TREN, which was added after degassing), solvent, CuBr₂ (if used in the reaction) and CuBr were added to a Schlenk flask and degassed by three freeze-pump-thaw cycles. After stirring the mixture at room temperature for 1 hour the initiator, (an initiator without the group required for attachment to the Fc face of a MAb), EtBriBu, was added to start the reaction. The polymerization was stopped by opening the flask and exposing the catalyst to air. The reaction mixture was then diluted with methylene chloride and passed through a column filled with neutral alumina to remove the copper complex. The remaining unreacted PEOMA macromonomer was removed by ultrafiltration in MeOH/THF (50/50 vol. %) solution mixture, and the pure brush polymer was dried under vacuum to a constant weight.

In the case of the RAFT experiment, PEOMA (2.02 g, 1.84 mmol), CDB (0.002 g, 0.0074 mmol) and anisole (1.5 ml) were combined in the Schlenk flask, degassed and then the initiator, AIBN (0.00024 g, 0.0015 mmol) dissolved in 0.5 ml anisole (after bubbling by nitrogen for 10 min. at 0° C.), was added.

Preparation of the Precursor of the Macroinitiator for “Grafting From” by Polymerization of HEMA-TMS (DPₙ=400): (Exemplary of Step 2a, but targeting a high MW polymer segment):

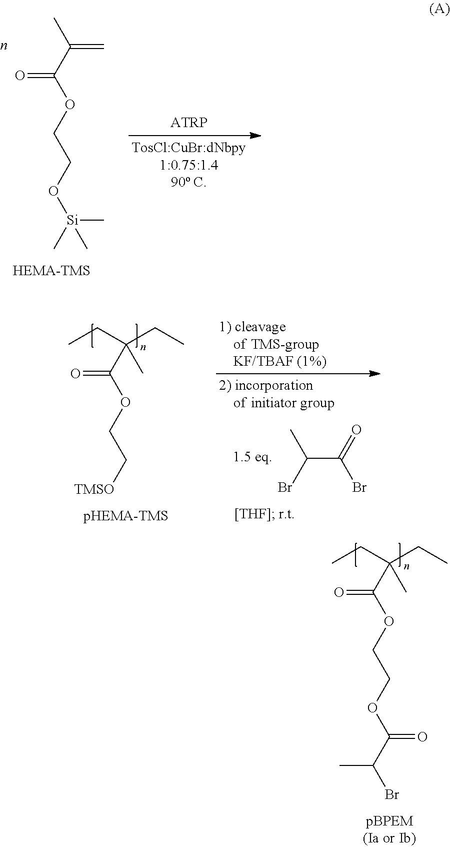

Example 2a-1 TosCl (0.0095 g, 0.05 mmol), 1 ml HEMA-TMS and anisole (0.54 ml, 10 vol. %) were combined in a 10 ml round bottom flask, and argon was bubbled through the solution for 10 min at 0° C. dNbpy (0.0306 g, 0.075 mmol) and the rest of monomer (4.4 ml) were combined in a 25 ml Schlenk flask and degassed by three freeze-pump-thaw cycles then CuBr (0.0054 g, 0.0375 mmol) was added. After stirring the mixture for 10 min to form the catalyst complex, the Schlenk flask was placed in a thermostated oil bath set at 90° C. After 3 min the initiating system was transferred into the Schlenk flask and an initial sample was taken. During polymerization, samples were periodically removed to determine molecular weight of the polymer by GPC and analyze conversion by GC. The reaction mixture was stirred for a total of 5.5 h, before being cooled to ambient temperature, exposed to air, diluted with CH₂Cl₂ and filtered through a neutral alumina column to remove the copper catalyst. Finally, the solvent was removed and the polymer was dried under vacuum to a constant weight.

### Example 2a-2

### (2-(2-Bromopropionyloxy)ethyl Methacrylate (pBPEM) (Ia)

PHEMA-TMS (Mn) 1.01_{—}105, Mw/Mn) 1.12) (9.9 g, assumed 49 mmol) was dissolved in 125 mL of dry THF under nitrogen. Potassium fluoride (2.85 g, 49 mmol) was added followed by slow addition of 0.5 mL of tetrabutylammonium fluoride (1 M in THF; 0.5 mmol) and then dropwise addition of 7.75 mL of 2-bromopropionyl bromide (74 mmol) over the course of 15 min. The reaction mixture was stirred at room temperature for 4 h, exposed to air, precipitated into methanol/ice (50/50 v/v), dissolved in 200 mL of CHCl₃, and filtered through an activated alumina column (basic). The polymer was reprecipitated three times in hexanes and dried in a vacuum oven at 25° C. for 24 h. 8.7 g of pBPEM was obtained (83% yield).

Transformation of P(HEMA-TMS) to PBPEM: (Exemplary of Step 2b):

P(HEMA-TMS)(M_{n,app}=81×10³ g/mol; M_{w}/Mₙ=1.19) (10 g; assumed 50 mmol) was dissolved in 125 ml dry THF under nitrogen. Potassium fluoride (2.9 g; 50 mmol) was added followed by slow addition of 0.5 ml tetrabutylammonium fluoride (1M in THF; 0.5 mmol) and then dropwise addition of 7.8 ml 2-bromopropionyl bromide (75 mmol) over the course of 15 minutes. The reaction mixture was stirred at room temperature for 4 h, exposed to air, precipitated into methanol/ice (50/50 v/v), dissolved in 200 ml CHCl₃ and filtered through activated alumina column (basic). The polymer was reprecipitated three times in hexanes and dried in vacuum oven at 25° C. for 24 h.

P(BPEM-graft-p(nBuA)): (Exemplary of Step 3; formation of the core of the nano-device).

In a 100 mL Schlenk flask pBPEM (0.3 g, 1.14 mmol initiator centers), CuBr2 (0.0063 g, 0.028 mmol) and dNbpy (0.463 g, 1.14 mmol) were purged three times with inert gas. Deoxygenated nBuA (58.5 g, 65.14 mL, 0.46 mol) and MEK (3 mL, 4 vol %) were then added, and the reaction mixture was degassed by three freeze-pump-thaw cycles. After stirring for 1 h at rt, CuBr (0.082 g, 0.568 mmol) was added, an initial sample was taken, and the flask was placed in a thermostated oil bath at 70° C. During the polymerization, samples were removed to analyze conversion by GC using MEK as the internal standard. The polymerization was stopped at 3.5% conversion (GC) after 12 h by cooling to rt and opening the flask to air. GPC-MALLS analysis was proceeded with a crude polymer sample (Mn)) 0.97×10⁶, Mw/Mn) 1.22), and after purification of the polymer following the procedure described above (precipitation in methanol with 20 vol % water), 2.72 g was obtained as isolated polymer (4.3% monomer conversion).

Using the same procedure, pBPEM-graft-pnBuA was prepared by grafting nBuA from pBPEM. The polymerization was stopped after 26 h at 14% (GC) monomer conversion (12% gravimetry). Mn) 3.4×10⁶ and Mw/Mn) 1.38 were determined by GPC-MALLS analysis.

The synthesis of an exemplary functional initiator suitable for attachment to a protein. (J. Am. Chem. Soc. 2004, 126, 15372-15373.)

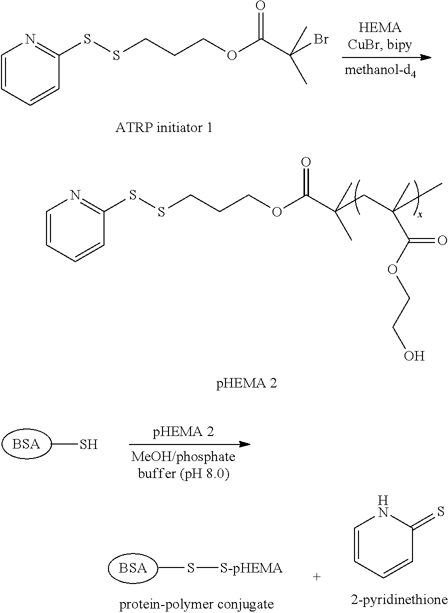

Hydroxypropyl-mercaptopyridine (0.50 g, 2.5 mmol), 1,3-dicyclohexylcarbodiimide (DCC, 0.516 g, 2.5 mmol) and 4-dimethylaminopyridine (DMAP, 0.031 g, 0.25 mmol) were dissolved in 20 mL of dry dichloromethane. 2-Bromo-2-methylpropionic acid (0.414 g, 2.5 mmol) was added and the reaction mixture was stirred overnight at room temperature. The reaction mixture was filtered, the solvent evaporated under vacuum and the oily residue purified by column chromatography (hexanes:ethyl acetate 60:40) to give compound 1. Yield: 53%. 1H NMR (CDCl₃, 500 MHz): δ 8.47 (m, 1H), 7.67 (m, 2H), 7.10 (m, 1H), 4.29 (t, 2H, J=6.05 Hz), 2.91 (t, 2H, J=7.16 Hz), 2.11 (m, 2H), 1.91 (s, 6H). 13C NMR (d6-DMSO): δ 170.99, 159.23, 149.95, 138.12, 121.56, 119.65, 64.16, 57.62, 34.53, 30.45, 27.80. HRMS (EI): calcd for (M)+: 348.9806; found 348.9823.

Polymerization from Initiator 1.

In a typical example, a Schlenk tube was charged with CuBr (12 mg, 0.084 mmol) and bipy (26 mg, 0.166 mmol) and evacuated-argon refilled three times. Degassed d4-methanol (0.50 mL) and HEMA (0.50 mL, 4.12 mmol) were transferred into the Schlenk tube. Degassed 1 (29 mg, 0.082 mmol) was added to start the reaction. The mixture was stirred at room temperature. For kinetic experiments, samples were periodically removed and diluted in DMF (for GPC) or d4-methanol (for 1H NMR). To stop the reactions, the polymerization solutions were diluted with methanol and filtered over silica. The polymers were isolated by precipitation in cold diethyl ether. Precipitation was repeated a minimum of 2 times. The degree of polymerization was obtained from the 1H NMR spectra by comparing the integrations resulting from the methylene protons adjacent to the hydroxyl functionality in the pHEMA side chains with the methylene protons adjacent to the disulfide. Residual monomer in the kinetic experiments was taken into account by subtracting the integration resulting from the olefinic proton at 5.6 ppm from I.

Reduction of pHEMA 2 with Dithiothreitol (DTT) and Determination of the End Group Content.

pHEMA 2 (1.98 mg) was dissolved in a minimal amount of methanol and then diluted with phosphate buffered saline (PBS, pH 7.3) to 5 mL. 1 mL of this solution was mixed with 1 mL of a DTT solution (1.20 mM in PBS pH 7.3). The absorbance at 343 nm was determined after 10 and 30 minutes. The concentration of the released 2-pyridinethione was calculated using the reported molar extinction coefficient (8.08×10 3 M-1 cm-1).2 Comparison with the total polymer concentration (molecular weight determined from 1H NMR) gave the end group percentage.

Reaction of pHEMA 2 with N-acetyl-L-cysteine and Determination of the End Group Content.

pHEMA 2 (2.11 mg) was dissolved in 5 ml of methanol. 1 ml of this solution was mixed with 1 mL of a 0.985 mM solution of N-acetyl-L-cysteine in water. After 1 hour of reaction, the absorbance at 343 nm was recorded. The concentration of the released 2-pyridinethione was calculated using a calibration curve obtained by reacting 1.04 mM N-acetyl-L-cysteine (in water) with increasing concentrations of 2,2′-dithiopyridine in methanol. The values obtained for the release of 2-pyridinethione from pHEMA 2 by reaction with N-acetyl-L-cysteine were consistent with those obtained after treatment with DTT.

Conjugation of pHEMA to BSA. (Exemplary of One Method of Attachment to a Bio-Responsive Molecule)

A solution of pHEMA 2 (˜50 eq to BSA) in methanol (4.5 mL) was added to a solution of BSA (10 mg in 5 mL of phosphate buffer, pH 8.0). The mixture was incubated for 30 minutes at room temperature. A small aliquot was immediately used for SDS-PAGE analysis after evaporation of the methanol. The remaining solution was dialyzed (MWCO=50,000), lyophilized, and used for the Ellman's assay.

All references cited herein are hereby incorporated by reference in their entirety. Though the embodiments have been described in various combinations to help describe the invention, elements of one embodiment may be combined with other elements of another embodiment to produce functional drug delivery devices of the invention.

### REFERENCES

### Patents

Wooley, K. L., et. al. U.S. patent application Ser. No. 11/250,830 published as 20060159619.
Mirkin, C. A. et. al., U.S. patent application Ser. No. 11/050,983 published as 20060068378.
Schwarz, M. C.; Richard, R. E.; Zhong, S.-P. (Boston Scientific Scimed, Inc., USA) In PCT Int. Appl.; WO/2005113031, 2005, p 26 pp.
Matyjaszewski, K. et al.; (Carnegie Mellon University) U.S. Pat. Nos. 7,125,938; 7,064,166; 7,056,455; 7,049,373; 7,019,082; 6,887,962; 6,790,919; 6,759,491; 6,627,314; 6,624,263; 6,624,262; 6,541,580; 6,538,091; 6,512,060; 6,407,187; 6,162,882; 6,124,411; 6,121,371; 6,111,022; 5,945,491; 5,807,937; 5,789,487; and 5,763,548.

### Papers

Shire, S. J.; Shahrokh, Z.; Liu, J. J. Pharm. Sci. 2005, 93, 1390-1402.
Daugherty, A. L.; Mrsny, R. J. Adv. Drug Deliv Rev. 2006, 58, 686-706.
Liu, J.; et al. J Pharm Sci. 2005, 94, 1928-1940.
Rosenberg, A. S. AAPS J. 2006, 8, E501-E507.
Hamann, P. R.; et. al., Bioconjugate Chem. 2005, 16, 354-360.
Ferrari, M., et. al., Nature Reviews Cancer, 2005, 5, 161.
Chari, R. V. J. Acc. Chem. Res. 2008, 41, 98-107.
Polakis, P.; Current Opinion in Pharmacology, 2005, 5(4), 382-387.
Wooley, K. L., et. al., Macromolecules 2004, 37(19), 7109-7115.
Mirkin, C. A. et. al., Chem. Rev., 2005, 105, 1547-1562.
Fréchet, J. M. J. et. al., Nature Biotech., 2005, 23, 1517-26.
Fréchet, J. M. J. et. al., PNAS. 0607705103.
Braunecker, W. A.; Matyjaszewski, K. Progress in Polymer Science 2008, 33, 165.
Sciannamea, V.; Jerome, R.; Detrembleur, C. Chem. Rev. 2008, 108, 1104-1126.
Moad, G.; Rizzardo, E.; Thang, S. H. Polymer 2008, 49, 1079-1131.
York, A. W.; Kirkland, S. E.; McCormick, C. L. Adv. Drug Delivery Rev. 2008, 60, 1018-1036.
Blainey, J. D. Curr. Probl. Clin. Biochem. 1968, 2, 85.
Froidevaux, S.; et al. J Nucl Med. 2004, 45, 116-123.
Allen, B. J. Rev. Recent Clin. Trials 2008, 3, 185-191.
Kelly, M. P.; et.al; Cancer Biother. Radiopharm. 2008, 23, 411-424.
Ali, M.; Brocchini, S. Advanced Drug Delivery Reviews 2006, 58, 1671-1687.
Burke, S. E.; Kuntz, R. E.; Schwartz, L. B. Advanced Drug Delivery Reviews 2006, 58, 437-446.
McCormick, C. L.; Kirkland, S. E.; York, A. W. Polymer Reviews, 2006, 46, 421-443.
Moad, G.; Mayadunne, R. T. A.; Rizzardo, E.; Skidmore, M.; Thang, S. H. Macromolecular Symposia 2003, 192, 1-12.
J. S. Wang, K. Matyjaszewski, J. Am. Chem. Soc., 1995, 117, 5614.
Matyjaszewski, K.; Xia, J. Chem. Rev. 2001, 101, 2921-2990.
Tsarevsky, N. V.; Bencherif, S. A.; Matyjaszewski, K. Macromolecules 2007, 40, 4439-4445.
Gao, H. and K. Matyjaszewski (2008) Macromolecules 41(12): 4250-4257.
McCarthy, P., et al. (2006) ACS Symposium Series 944: 252-268.
Matyjaszewski, K.; et. al., J. Am. Chem., Soc. 2006, 128, 5578.
Matyjaszewski, K.; et. al., Macromolecules 2004, 37, 9768.
Maynard, H. D.; et. al.; J. Am. Chem. Soc. 2004, 126, 15372-15373
Matyjaszewski, K.; et. al., ACS Symp. Ser. 2006, 937, 79.
Matyjaszewski, K.; et. al., Polymer 2005, 46, 11698.
Matyjaszewski, K.; et. al., Macromol. Rapid Commun. 1997, 18, 1057-1066.
Tsarevsky, N. V.; Matyjaszewski, K. Macromolecules 2002, 35, 9009-9014.
Beers, K. L.; et. al., Macromolecules 1998, 31, 9413-9415.
Beers, K. L.; Boo, S.; Gaynor, S. G.; Matyjaszewski, K. Macromolecules 1999, 32, 5772-5776.

1. A multi-functional nano-device that can be conjugated to a single bio-responsive agent wherein the multi-functional nano-device comprises a core shell brush structure.

2. The multi-functional nano-device of claim 1, wherein the conjugated bio-responsive agent is a monoclonal antibody or a fragment thereof.

3. The conjugated multi-functional nano-device of claim 2, wherein the multi-functional nano-device is conjugated to the Fc of the monoclonal antibody through a single oligo/polymer link.

4. The multi-functional nano-device of claim 1, wherein the nano-device is prepared by a controlled polymerization process and the dimensions of the nano-device are determined by the degree of (co)polymerization of each segment of the nano-device.

5. The multi-functional nano-device of claim 1, wherein the phylicity of the core and the shell differ.

6. The multi-functional nano-device of claim 5, wherein the core comprises one or more hydrophobic monomer units and the shell comprises one or more hydrophilic monomer units.

7. The multi-functional nano-device of claim 1, wherein the core of the nano-device can be loaded with therapeutic agents.

8. The loaded multi-functional nano-device of claim 7, wherein the core of the nano-device comprises one or more incorporated therapeutic agents.

9. The loaded multi-functional nano-device of claim 8, wherein the core of the nano-device can be loaded with one or more small molecule drug agents.

10. The loaded multi-functional nano-device of claim 8, wherein the core of the nano-device can be loaded with one or more magnetic or image enhancing agents.

11. The multi-functional nano-device of claim 1, wherein the core of the nano-device can accommodate on average more than 5 individual therapeutic molecules.

12. The multi-functional nano-device of claim 11, wherein the core of the nano-device can accommodate on average more than 10 small molecule drug molecules.

13. The multi-functional nano-device of claim 7, wherein the therapeutic agents are held within the core of the nano-device through non-covalent interactions.

14. The multi-functional nano-device of claim 1, wherein the core is attached to the shell through bio-responsive functional linking groups.

15. The multi-functional nano-device of claim 14, wherein the shell of the nano-device can be cleaved from the core of the nano-device in the presence of a cancerous environment.

16-20. (canceled)

21. The multi-functional nano-device of claim 2, wherein the monoclonal antibody is selected to transport the nano-device to a particular cancer cell line.

22. The multi-functional nano-device of claim 21, further comprising a specific fluorophore unit that can be associated with a specific MAb is selectively conjugated to the selected MAb.

23. The multi-functional nano-device of claim 22, wherein the cleaved hydrophilic shell fragments allow identification of the presence of the first specific MAb targeted cell line in a living body.

24. A mixture of the multifunctional nano-devices of claim 22 whereby the presence of a particular cleaved hydrophilic shell fragments allows identification of the presence of one or more specific MAb targeted cell line(s) in a living body.

25. The multifunctional nano-device of claim 1 wherein an additional non-drug encapsulated functionality is incorporated into the core of the nano-device conjugate and when the nano-device conjugated to a series of MAb's detects specific cancerous environments within the body and the cleaved urine detectable unit informs the patient/doctor that a specific cancer is present the further encapsulated functionality is clearly seen in subsequent MRI screening tests.

26-58. (canceled)
